(19) United States
(12) Patent Application Publication
(10) Pub. No.: US 2024/0138545 A1
(43) Pub. Date: May 2, 2024

(54) COSMETIC USE OF A DEVICE EMITTING MILLIMETER WAVE RF RADIATION

(71) Applicant: REMEDEE LABS, MONTBONNOT-SAINT-MARTIN (FR)

(72) Inventors: Virginie PIERRE, GRENOBLE (FR); David CROUZIER, MEYLAN (FR); Michael FOERSTER, CORENC (FR)

(21) Appl. No.: 18/548,862

(22) PCT Filed: Mar. 3, 2022
(86) PCT No.: PCT/EP2022/055368

§ 371 Date: Sep. 1, 2023

Related U.S. Application Data
(60) Provisional application No. 63/156,104, filed on Mar. 3, 2021.

(51) Int. Cl. A45D 44/00; A61N 5/02

(52) U.S. Cl. CPC A45D 44/002; A61N 5/025; A45D 2200/205

(57) ABSTRACT

The invention relates to the cosmetic use of a device for preventing and/or reducing the signs of skin aging and/or the resultant appearance of aged skin, and/or for preventing and/or reducing the signs of cutaneous stress, wherein said device is capable of transmitting electromagnetic waves having a power flux density of at least 0.5 mW/cm² to skin and a frequency value of between 30 and 90 gigahertz. It also relates to a cosmetic process for preventing and/or reducing the signs of skin aging and/or the resultant appearance of aged skin, and/or for preventing and/or reducing the signs of cutaneous stress, which comprises the use of a device comprising a detection unit of human or animal skin, and a transmitter, said process comprising the following steps: a) the detection unit detects human or animal skin, and b) when the unit detects that the skin is located at three millimeters or less from the transmitter, the transmitter transmits the electromagnetic waves, wherein said waves have a power flux density of at least 0.5 mW/cm² to skin and a frequency value of between 30 and 90 gigahertz.

### TECHNICAL FIELD

The invention relates to the cosmetic treatment of skin and, more particularly, to the cosmetic use of a device for preventing and/or reducing the signs of skin aging and the resultant appearance of aged skin, and/or for preventing and/or treating the signs of cutaneous stress.

### BACKGROUND

Human skin is comprised of three layers. Respectively from the surface inwards, the three layers are known as the epidermis, the dermis and the hypodermis. Each of these three layers has a thickness that varies according to an individual's gender and age, as well as the bodily location of the skin in question. The epidermis generally is between about 50 and about 150 μm thick (see the publication by Lewis, J., Raff, M., Roberts, K., Watson, JD, Alberts, B., Bray. D., “Molecular Biology of the Cell,” Vol. 3. Garland Publisher (2009)). The dermis is, on average, about 2 mm thick with the average thickness varying from about 300 μm on an individual's eyelids to about 3 mm on an individual's back (see the publication by Ebling, R., “Wilkinson Textbook of Dermatology,” Vol. 5, Blackwell Scientific Publications (2009)). The thickness of the hypodermis can vary greatly among individuals and generally is the greatest in the buttocks, on the palms of the hands, and on the soles of the feet.

The epidermis mainly is comprised of keratinocytes and nourished from the dermis. The dermis is connected to the epidermis by a stratum basal layer comprised of basal cells, precursor cells to the keratinocytes of the epidermis. The dermis contains ultra-structures in the form of mechano-receptors and thermo-receptors, hair follicles, various glands, blood and lymphatic vessels. The dermis is mainly comprised of fibroblasts and an extracellular matrix which is synthetized by fibroblasts. The extracellular matrix primarily is comprised of collagen, elastic fibers and an extrafibrillar matrix comprising proteoglycans (see the following publications: Ebling R., “Wilkinson Textbook of Dermatology,” Vol. 5. Blackwell Scientific Publications (2009)); Geronemus, R G, “Fractional Photothermolysis: Current and Future Applications,” Lasers Surg. Med., 38: 169-176 (2006)). The hypodermis mainly is comprised of fibroblasts like the dermis, but with less connective tissue. The hypodermis also comprises fat cells.

With age, human skin loses thickness, becoming thinner and more easily damaged. The skin's capacity for healing and regeneration also decreases. Thus, aging of the skin may be characterized by a decrease in its volume and elasticity.

Several cosmetic treatments are available which are intended to prevent and/or reduce the aging of the skin (and the resultant appearance of aged skin). Some such treatments aim to transfer energy to the deep layers of the skin, such as the dermis. Such treatments include radiation of the skin with infrared (“IR”) radiation and/or radiofrequency (“RF”) radiation. IR radiation may be divided into Infrared Radiation A (“IRA,” having a wavelength of 760-1440 nanometers), Infrared Radiation B (“IRB,” having a wavelength of 1440-3000 nanometers) and Infrared Radiation C (“IRC,” having a wavelength of 3000 nanometers−1 millimeter). Of the categories of IR radiation, only IRA is able to penetrate deep layers of the skin, such as the dermis.

IR radiation has been used in cosmetic dermatology to treat wrinkles and sagging skin by stimulating the production of collagen I, collagen III, and elastin (see the publication by Tanaka Y., Matsuo K., Yuzuriha S., “Long-Term Evaluation of Collagen and Elastin Following Infrared (1100 to 1800 nm) Irradiation,” J. Drugs Dermatol., 8:708-12 (2009) [https://pubmed.ncbi.nlm.nih.gov/19663107/]).

Radiofrequency (RF) radiation therapy can be ablative or non-ablative. Non-ablative RF radiation has been used for the treatment of skin laxity (see the publication by Araújo A R, Soares V P C, Silva F S, Moreira T S, “Radiofrequency for the Treatment of Skin Laxity: Myth or Truth,” An Bras Dermatol., 90(5):707-21 (2015)) implying a controlled rise in tissue temperature from a high frequency alternating current (0.3 to 10 MHz). The rising of temperature and the depth of heating depend on the level of energy used and on the impedance of the biological tissue being treated (see the publication by Belenky I., Margulis A., Elman M., Bar-Yosef U., Paun S D, “Exploring Channeling Optimize Radiofrequency Energy: A Review of Radiofrequency History and Applications in Esthetic Fields,” Adv. Ther.; 29:249-66 (2012)). The objective of such treatments is to produce a thermal shock responsible for a change in the conformation of collagen, as well as for stimulating the production of new collagen (see the publication by Alster, T S, Jason, R L, “Nonablative Cutaneous Remodeling Using Radiofrequency Devices,” Clin. Dermatol., 25:487-91 (2007)).

### SUMMARY OF THE INVENTION

Thus, it is an object of the invention to use electromagnetic waves (or radiofrequency (“RF”) radiation) in the cosmetic field. Especially, it is an object of the invention to use electromagnetic waves to prevent and/or reduce the signs of skin aging and/or the resultant appearance of aged skin and/or to prevent and/or treat the signs of cutaneous stress. An object of the invention is to provide devices for the administration of electromagnetic waves to human skin, the devices being effective for applying the electromagnetic waves to human skin in selected areas, for instance, areas that often are visible on an individual and that, accordingly, many individuals desire for them to show limited signs of aging, if any. Such areas may include, by way of example only, the face, the hands, the décolletage, the eye region, the lips, and the neck.

To this end, the invention provides devices that selectively provide electromagnetic waves to targeted portions of a human individual's skin. Such devices may include technology that permits the devices to act as heat skin stressors at a specific depth of penetration without having a damaging impact on the skin. The cosmetic use of said devices allows preventing and/or reducing the aging of skin. The cosmetic use of said devices allows preventing and/or reducing the appearance of skin wrinkles and/or fine lines.

Thus, the present invention relates to the cosmetic use of a device for preventing and/or reducing the signs of skin aging and/or the resultant appearance of aged skin, and/or to prevent and/or treat the signs of cutaneous stress, wherein said device is capable of transmitting electromagnetic waves having a power flux density of at least 0.5 mW/cm² to skin and a frequency value of between 30 and 90 gigahertz. Preferably the device is capable of simultaneously exposing at least 1.25 cm² of the skin to the waves. Preferably, said cosmetic use is for preventing and/or reducing the appearance of skin wrinkles and/or fine lines. Preferably, the waves have a power flux density of between 0.5 and 15 mW/cm², preferably between 3 and 12 mW/cm², preferably between 5 and 10 mW/cm². Preferably, the waves have a frequency value of between 60 and 80 gigahertz. Preferably the skin is human skin. Preferably the device is used for at least a few minutes, preferably at least 20 minutes. Preferably the device is used for a few hours, preferably from 20 minutes to 2 hours, preferably from 30 minutes to 1 hour. The device may increase the skin temperature both through conducted heat transfer and through the absorption into the skin of electromagnetic wave radiation.

The present invention also relates to a cosmetic process for preventing and/or reducing the signs of skin aging and/or the resultant appearance of aged skin, and/or for preventing and/or treating the signs of cutaneous stress, which comprises the use of a device comprising a detection unit of human or animal skin, and a transmitter, said process comprising the following steps:
a) the detection unit detects human or animal skin, and
b) when the unit detects that the skin is located at three millimeters or less from the transmitter, the transmitter transmits the electromagnetic waves, wherein said waves have a power flux density of at least 0.5 mW/cm², preferably between 0.5 and 15 mW/cm², preferably between 3 and 12 mW/cm², preferably between 5 and 10 mW/cm², to skin and a frequency value of between 30 and 90 gigahertz.

Preferably, the device is suitable for applying electromagnetic waves to human skin in selected areas. Preferably, the device is suitable for applying electromagnetic waves to human skin in at least one area chosen from the face, the hands, the décolletage, the eye region, the lips and the neck.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 is a schematic diagram depicting the combined effect of both conductive heating and RF heating for increasing the temperature of the dermis without a corresponding excessive temperature increase of the epidermis according to embodiments of the disclosure;

FIG. 2 is a schematic diagram depicting an exemplary stimulation pattern comprising durations of stimulation and durations of idle time according to embodiments of the disclosure;

FIG. 3 is a schematic diagram depicting different areas on the human face that can be prone to wrinkles and/or other signs of aging according to embodiments of the disclosure;

FIG. 4 is a schematic diagram depicting a module control unit in communication with a facemask having a plurality of modules disposed on an inside surface thereof that are designed to be stimulated sequentially according to embodiments of the disclosure;

FIG. 5 is a schematic diagram illustrating an exemplary device wherein each millimeter wave module includes multiple ASICs according to embodiments of the disclosure.

FIG. 6 is an exemplary device suitable for administering electromagnetic waves to a human face according to embodiments of the disclosure;

FIG. 7 is an exemplary device suitable for administering millimeter wave RF stimulation to a human face according to embodiments of the disclosure;

FIG. 8 is an exemplary device suitable for administering millimeter wave RF stimulation to the neck and décolletage according to embodiments of the disclosure;

FIG. 9 is an exemplary device suitable for administering millimeter wave RF stimulation to human lips according to embodiments of the disclosure;

FIG. 10 is an exemplary device suitable for administering millimeter wave RF stimulation to human hands according to embodiments of the disclosure;

FIG. 11 is an exemplary device suitable for administering millimeter wave RF stimulation to human hands according to embodiments of the disclosure;

FIG. 12 is an exemplary device suitable for administering millimeter wave RF stimulation to the eye region of a human face according to embodiments of the disclosure;

FIG. 13 illustrates a flow chart of an example process for preventing and/or reducing the aging of human skin and, accordingly, the resultant appearance of aged skin by utilizing a device having means for administering electromagnetic waves according to certain aspects of the disclosure; and

FIG. 14 illustrates an electronic system with which one or more implementations of the subject technology may be implemented.

### DETAILED DESCRIPTION

In the present invention, radiofrequency (“RF”) radiation is a synonym for electromagnetic waves.

The invention relates to the use electromagnetic waves in the cosmetic field.

The present invention also relates to the cosmetic use of a device for preventing and/or reducing the signs of skin aging and/or the resultant appearance of aged skin, and/or for preventing and/or treating the signs of cutaneous stress, wherein said device is capable of transmitting electromagnetic waves having a power flux density of at least 0.5 mW/cm² to skin and a frequency value of between 30 and 90 gigahertz.

The claimed use may typically be performed at least once a week, preferably twice a week. Preferably, it is performed daily, typically once or twice a day. At every occurrence, preferably the device is used for at least a few minutes, preferably at least 20 minutes, and preferably at most a few hours, preferably from 20 minutes to 2 hours, preferably from 30 minutes to 1 hour.

The present invention also relates to a cosmetic process for preventing and/or reducing the signs of skin aging and/or the resultant appearance of aged skin, and/or for preventing and/or treating the signs of cutaneous stress, which comprises the use of a device comprising a detection unit of human or animal skin, and a transmitter, said process comprising the following steps:
a) the detection unit detects human or animal skin, and
b) when the unit detects that the skin is located at three millimeters or less from the transmitter, the transmitter transmits the electromagnetic waves, wherein said waves have a power flux density of at least 0.5 mW/cm², preferably between 0.5 and 15 mW/cm², preferably between 3 and 12 mW/cm², preferably between 5 and 10 mW/cm², to skin and a frequency value of between 30 and 90 gigahertz.

Preferably, the surface being human or animal skin, the device comprises a skin detection unit capable of signaling the presence or absence of the skin to be exposed to the waves, and, preferably, capable of determining a distance separating the skin and the device.

Thus, the device transmits waves directly towards the subject's skin only if the skin is detected. If the skin is not detected, or if the distance is too great between the device and the skin, no transmission takes place. This way, sending waves in any direction is avoided and energy is saved. The power or other parameters of the waves transmitted may also be adapted according to the estimated distance between the device and the skin.

The use and process according to the invention are designed for preventing and/or reducing the signs of skin aging and/or the resultant appearance of aged skin, and/or for preventing and/or treating the signs of cutaneous stress.

By “signs of skin aging”, it is meant any changes in the external appearance of the skin due to aging, whether chronobiological and/or photoinduced, such as for example wrinkles and fine lines, withered skin, lack of elasticity and/or skin tone, thinning of the dermis and/or breakdown of collagen fibers, resulting in the appearance of flabby, wrinkled skin. It also includes all the internal modifications of the skin which do not systematically result in a modified external appearance, such as for example all the internal degradations of the skin, particularly of the elastin fibers, or elastic fibers, consecutive to exposure to UV radiation. In particular, the cutaneous signs of aging targeted by the invention relate to all the modifications of the external appearance of the skin due to aging, whether chronological and/or photoinduced, such as for example a thinning of an epidermis and/or a loss of firmness, elasticity, density and/or tone of an epidermis and/or the formation of fine lines and wrinkles.

The cutaneous signs induced by stress («signs of cutaneous stress») can in particular result in sensations of cutaneous discomfort, sensory phenomena and/or the manifestation of undesirable, even painful, cutaneous signs such as irritation or a feeling of discomfort of the skin which can manifest in particular by tingling, tightness, overheating and/or itching. Also, it can be redness, itching, heating, burning sensations, tingling sensations or tightness. Other visible cutaneous signs may also appear, such as pruritus, scabs, inflammatory erythema, edema and/or pimples, or skin irritation reactions. Cutaneous stress may be due to external factors such as pollution (oxidative stress), temperature changes (heat or cold) or even various chemical agents with which the skin may be in contact (such as chlorine or cleaning products).

Brief exposure to temperatures exceeding the normal growth temperature of cells, as well as other stressors (e.g., exposure to UV light), are known to cause the generation of heat shock proteins (HSPs) in living organisms. When the exposed cells are skin cells, the HSPs are known to stimulate the regeneration and preservation systems of the skin. With the deep temperature rise, the lymphatic and blood vessels also are stimulated and skin permeability is increased.

Electromagnetic waves allow the transport of energy. When they are applied to biological tissue, they are absorbed by the tissue and they pass through to a maximum depth where all their energy has been dissipated. The maximum depth reached depends on their physical characteristics and the tissues they pass through. The energy absorbed by the tissues is transformed either into heat or chemical or electrical modification. Wavelength and electrical properties of the tissue are the key parameter of the depth penetration, and where the energy carried by the electromagnetic field is deposed.

The device of the invention preferably comprises a control module and a wave transmission module. In an embodiment, the wave transmission module, which can be called millimeter module (the waves being said to be “millimeter” in view of their wavelength) or millimeter card, measures 37 millimeters in length, 20 millimeters in width and is 3 millimeters thick. The millimeter module is able to transmit waves of frequency between 30 and 90 gigahertz, preferably between 60 and 80 gigahertz. The preferred frequency is 61.25 GHz+/−250 MHz. In all cases, the power flux density is at least 0.5 mW/cm², preferably between 0.5 and 15 mW/cm², preferably between 3 and 12 mW/cm², preferably between 5 and 10 mW/cm². Preferably the waves are transmitted simultaneously on a skin surface of 1.25 cm². Devices in accordance with embodiments of the invention typically emit (e.g., through antennas) millimeter wave radiation of about 61.25 GHz, plus or minus 250 MHz, at emitted power densities on the order of 5 to 10 mW/cm². Portions of the device also may increase in temperature, through the power consumption of the electronic components mounted on an electronic printed circuit incorporated therewith. The control module controls the transmission module. The control module may be activated by the subject, but it may also be programmed by the subject or another user, on the device directly with a button or via a terminal such as a computer.

At frequencies around 60 GHz, electromagnetic waves emitted by devices in accordance with embodiments of the invention may be able to penetrate bare human skin to depths of about 0.5 to 1 millimeter with about 90% of the power being absorbed in the epidermis and the dermis layer (see the publication by Wu, T., Rappaport, S., Collins, CM, “The Human Body and Millimeter-Wave Wireless Communication Systems: Interactions and Implications,” accepted in 2015 IEEE International Conference on Communications (ICC), June 2015). Most of the energy may be absorbed specifically at the location of fibroblasts in the skin, and at the stratum basal layer of the epidermis.

The steady state temperature elevation due to 10 W/m² at 60 GHz, on naked human skin, is low with an increase of about 0.16° C. due to the dissipated effect by blood flow in the muscle. In naked forehead skin, the temperature increase is about 0.3° C., likely due to the bone just beneath the skin having less blood flow and less thermal conduction capacity. The temperature elevation in naked forehead skin is low in the skin surface but high in the underlying tissues (SAT and bone) (see the publication by Wu, T., Rappaport, S., Collins, C M, “The Human Body and Millimeter-Wave Wireless Communication Systems: Interactions and Implications,” accepted in 2015 IEEE International Conference on Communications (ICC) (June 2015)).

Heating human skin utilizing a device in accordance with embodiments of the present disclosure may enhance capillary vasculature with an increase of arteriolar diameter. Such an increase in skin temperature may have the effect of improving transdermal penetration of various molecules. With the deep rise of skin temperature, the lymphatic and blood vessels may be stimulated and skin permeability increased (see the publication by Hao J., Ghosh P., Li S K, Newman B., Kasting G B, Raney S G, “Heat Effects on Drug Delivery Across Human Skin,” Expert Opin. Drug Deliv., 13(5):755-68 (2016)).

Temperature increase, in the proper range, also may increase the metabolism of the cells. As far as fibroblasts are concerned, the effect of the mild temperature increase may be an increase in collagen synthesis and an increase in cell proliferation and also increase cell viability (Siddiqui, S. H., Subramaniyan, S. A., Kang, D. et al., “Direct exposure to mild heat stress stimulates cell viability and heat shock protein expression in primary cultured broiler fibroblasts”. Cell Stress and Chaperones 25, 1033-1043 (2020)).

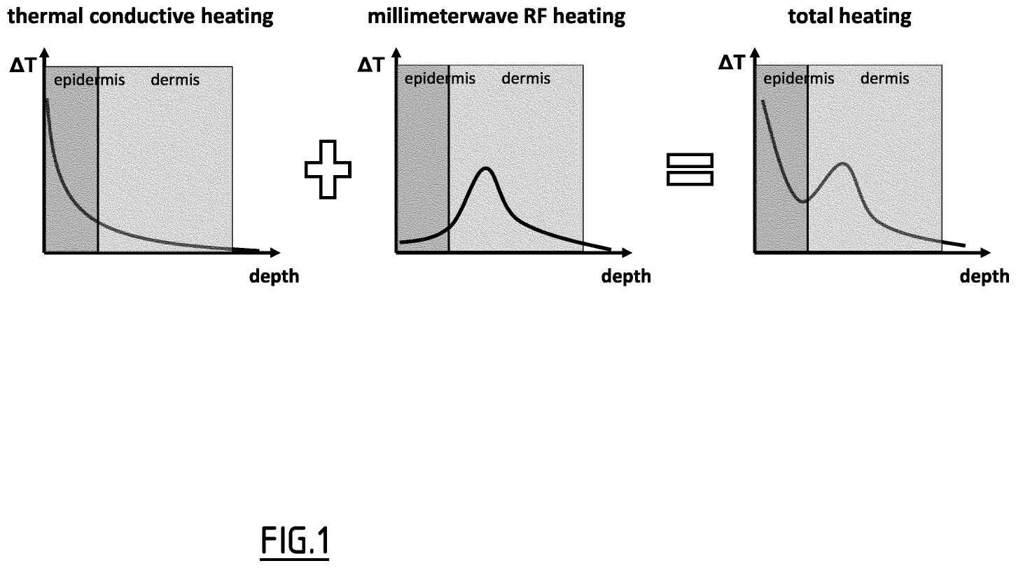

Devices in accordance with embodiments of the present disclosure may increase the skin temperature both through conducted heat transfer and through the absorption into the skin of millimeter wave RF radiation. The temperatures reached by the epidermis and dermis depend on the quantity and on the ratio of conducted heat and radiated heat transferred to the skin. The depth of absorption of the radiated heat depends on the RF frequency. The combined effect of both conductive heating and RF heating may allow an increase in the temperature of the dermis without a corresponding excessive (i.e., damaging) temperature of the epidermis while reaching the desired effect. The diagram of FIG. 1 visually illustrates this combined effect.

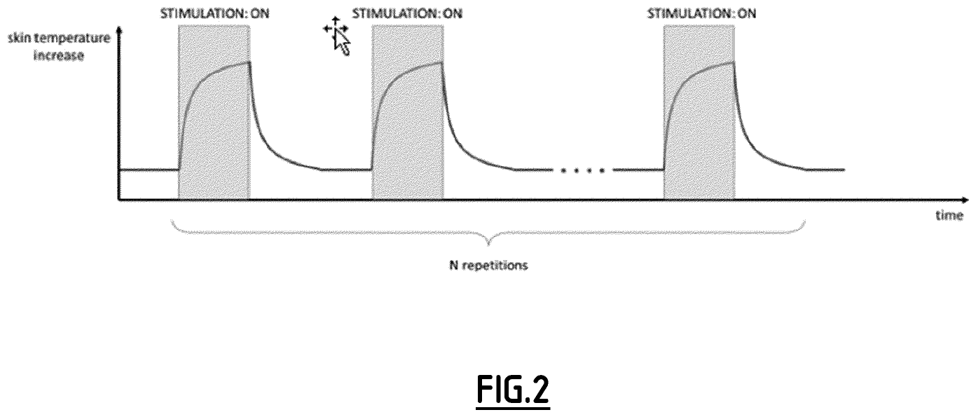

Specific heating patterns can be created through software in order to maximize the desired effect, where temperature increase, duration of heating, duration of idle time and number of repetitions are adjustable. One example of a stimulation pattern is illustrated in FIG. 2.

The best outcome in HSP overexpression appears to be reached when the targeted skin area is subject to a rise in body temperature (typically a few minutes) reaching a plateau that is held for a period (typically few tens of minutes) before allowing the skin to cool down again. Skin stress is known to stimulate the skin's regeneration and preservation systems as heat shock proteins (HSPs). The increase in HSP70 is able to protect the cells from further stress such as oxidative stress, heat and UV light (see the publications by Bellmann, K., Jaattela, M., Wissing, D., Burkart, V., Kolb, H., “Heat Shock Protein Hsp70 Overexpression Confers Resistance Against Nitric Oxide,” FEBS Letters, 391.1-2, 185-88 (1996); Li (1992); and Simon, M M, Reikerstorfer, A., Schwarz, A., Krone, C., Luger, T A, Jäättelä, M., “Heat Shock Protein 70 Overexpression Affects the Response to Ultraviolet Light in Murine Fibroblasts. Evidence for Increased Cell Viability and Suppression of Cytokine Release,” The Journal of Clinical Investigation, 95.3, 926-33 (1995)).

Induced by the transcription factor HSF-1 (Heat Shock Factor 1), HSPs proteins overexpression is able to act and prevent different kinds of damages (heat, oxidation, hypoxia . . . ) on cells and induce adaptive responses to stress and increase cell resistance to different types of stress. A study has shown the benefit on aged dermal fibroblasts in culture to repeated mild heat shocks (39-42.1° C.) with heat shock duration between 30 min and 1 hour (see the following publications: Rattan S I, “Repeated Mild Heat Shock Delays Ageing in Cultured Human Skin Fibroblasts,” Biochem. Mol. Biol. Int., 45: 753-759 (1998); Mayes A E, Holyoak C D, “Repeat Mild Heat Shock Increases Dermal Fibroblast Activity and Collagen Production,” Rejuvenation Res., 11: 461-465 (2008)). Alternatively pulsed heat shock of 2 seconds between 45° C. and 60° C. may be used. HSP90 is capable of inducing a re-programming of basal keratinocytes to initiate re-epithelialization (see the publication by Woodley, D T, Wysong, A., DeClerck, B., Chen, M., and Li, W., Advances in Wound Care, pp. 203-212 (April 2015).

HSPs proteins are able to link and stabilize the p53 protein. The p53 protein is a tumor suppressor factor and acts as a major barrier against cancer development. Native p53 helps maintain genome integrity and cellular homeostasis by regulating the expression of a plethora of genes involved in the regulation of cell cycle, apoptosis, stem cell differentiation, senescence, DNA repair, and metabolism (see the publication by Bieging K T, Mello S S, Attardi L D, “Unravelling Mechanisms of p53-Mediated Tumour Suppression,” Nat. Rev. Cancer 14:359-370 (2014)).

As millimeter waves increase temperature, HSP protein is stimulated and so positively acts upon p53. Millimeter wave stimulation also may maintain genome integrity.

The cosmetic process of the invention comprises the following steps:
a) the detection unit detects human or animal skin, and
b) when the unit detects that the skin is located at three millimeters or less from the transmitter, the transmitter transmits the electromagnetic waves, wherein said waves have a power flux density of at least 0.5 mW/cm², preferably between 0.5 and 15 mW/cm², preferably between 3 and 12 mW/cm², preferably between 5 and 10 mW/cm², to skin and a frequency value of between 30 and 90 gigahertz.

Preferably, said cosmetic process comprises, before step a), a step of determining the initial state of skin. Said step may be useful to determine whether the skin shows signs of skin aging and/or signs of skin stress. It may be a visual determination, or by using an apparatus designed for this purpose. Said step may also be useful to determine the parameters of step b) (notably power flux density and frequency of the electromagnetic waves).

Preferably, said cosmetic process comprises, after step b), a step c) of checking the final state of skin. Said step c) may be useful to determine whether step b) was beneficial, i.e. prevented and/or reduced the signs of skin aging and/or the resultant appearance of aged skin, and/or prevented and/or treated the signs of cutaneous stress. It may be a visual determination, or by using an apparatus designed for this purpose.

Preferably, said cosmetic process comprises, before or after step b) or after step c), the topical administration of a cosmetic product onto skin. Said cosmetic product may be any cosmetic product known in the art that is applicable onto skin, preferably a topical product. Preferably, the cosmetic product comprises at least one active agent chosen from hydrating agents, anti-ageing agents, firming agents and their mixtures. Hydrating agents may be chosen from humectants such as polyols and especially glycerol, alpha-hydroxy acids such as glycolic acid, and pyrrolidone carboxylic acid. Anti-ageing agents may be chosen from hyaluronic acid, vitamins such as retinol (vitamin A) or vitamin C. firming agents may be chosen from caffeine, resveratrol and coenzyme Q10.

Preferably, said cosmetic process comprises, in step b), means for adjusting the ratio of conducted heat and radiated heat transferred to the skin.

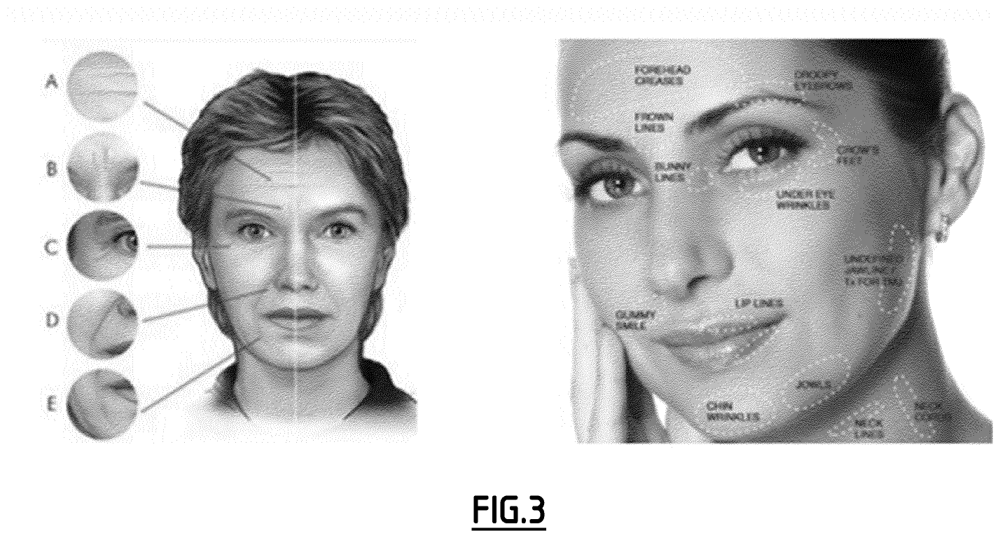

When seeking a local effect, it is necessary to cover the full surface area on which the outcome is desired, for instance different areas on the human face that can be prone to wrinkles and/or other signs of aging. FIG. 3 is a schematic diagram depicting several such areas on a human face. As can be seen, area A represents an area where human skin can be prone to forehead wrinkles, area B represents an area where human skin can be prone to frown lines, area C represents an area where human skin can be prone to crow's feet and/or under eye wrinkles, area D represents an area where human skin can be prone to what is known as “gummy smile,” and area E represents an area where human skin can be prone to jowls.

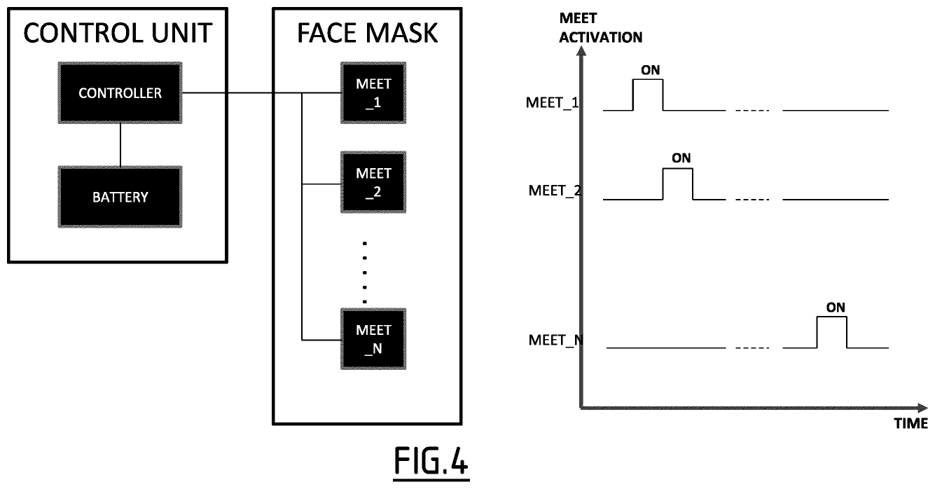

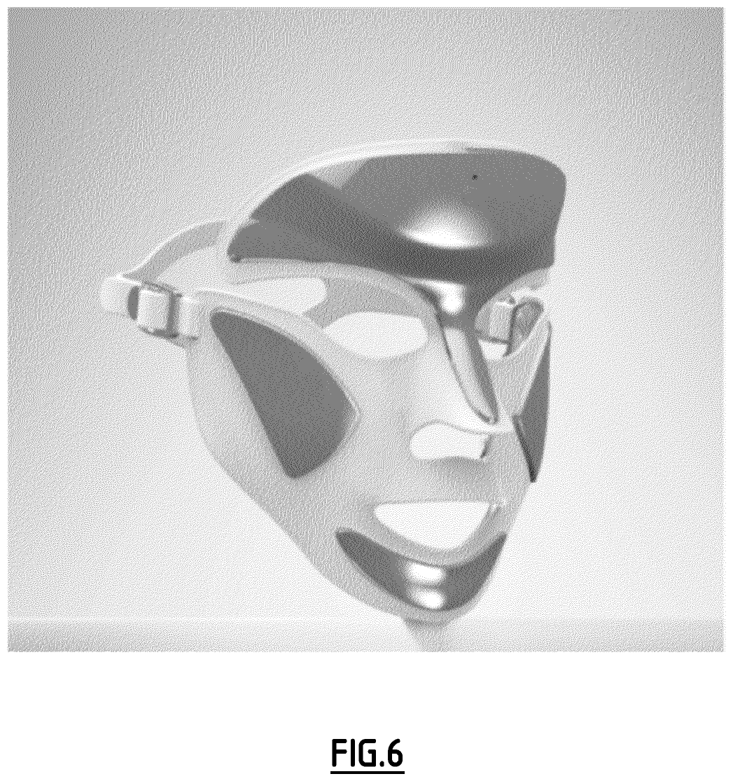

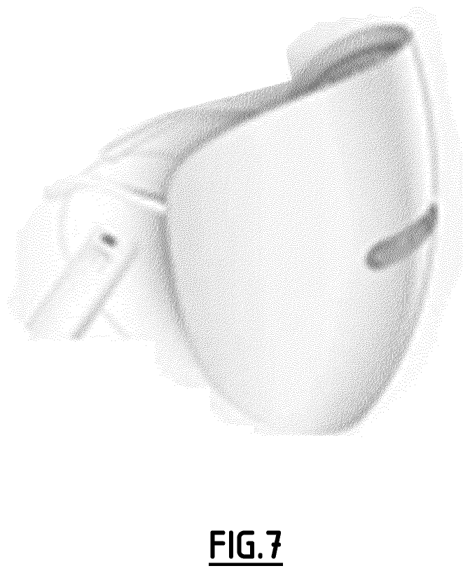

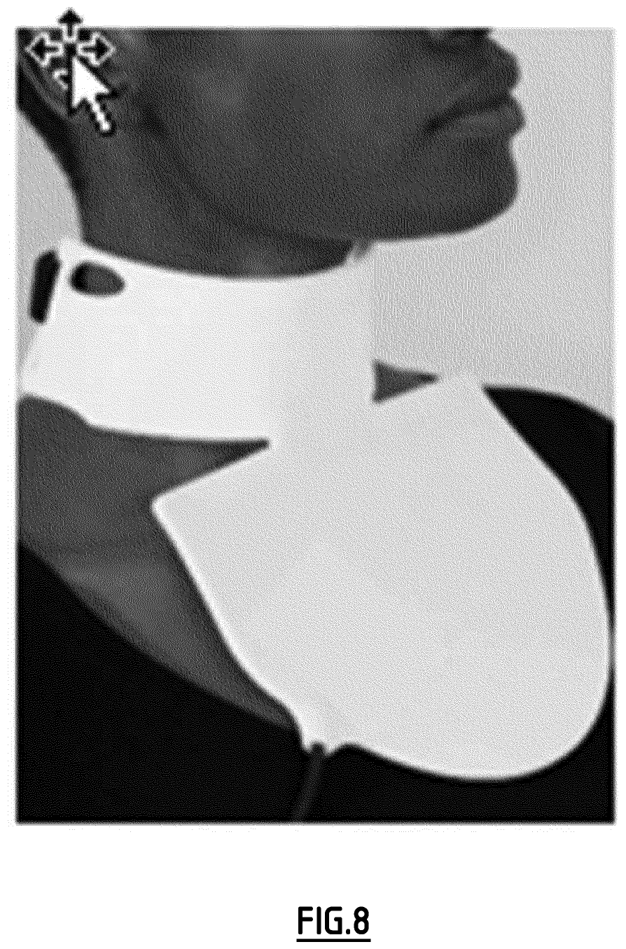

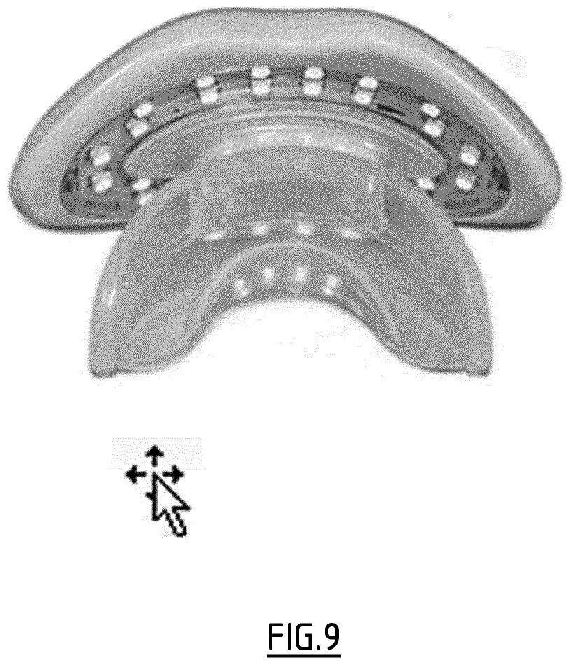

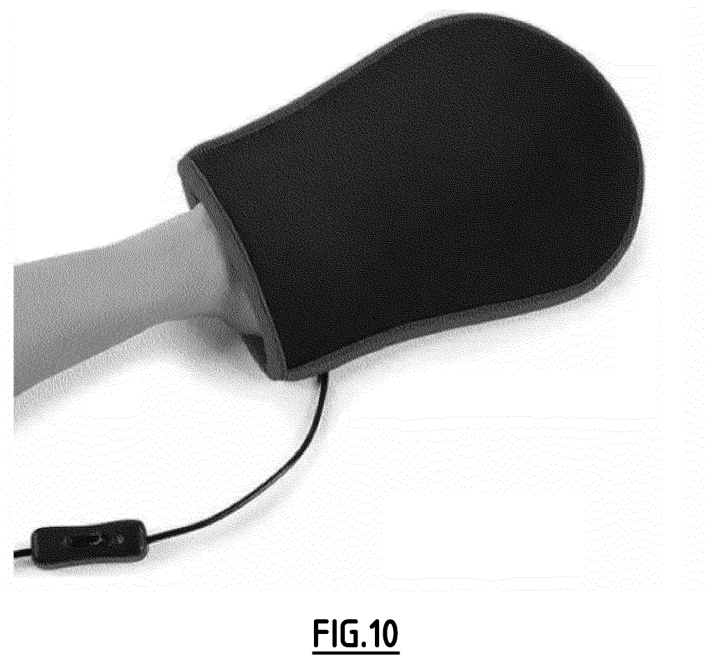

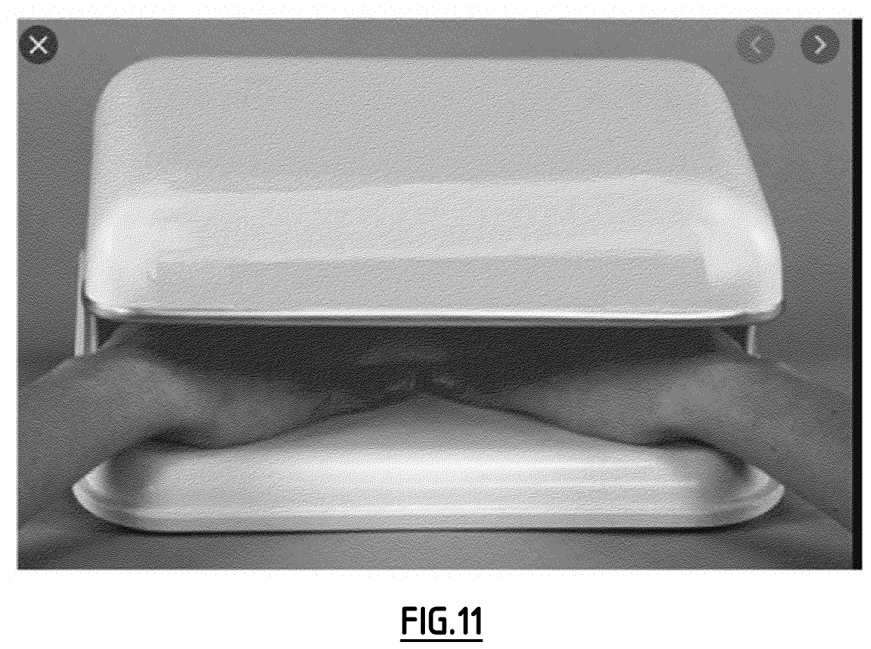

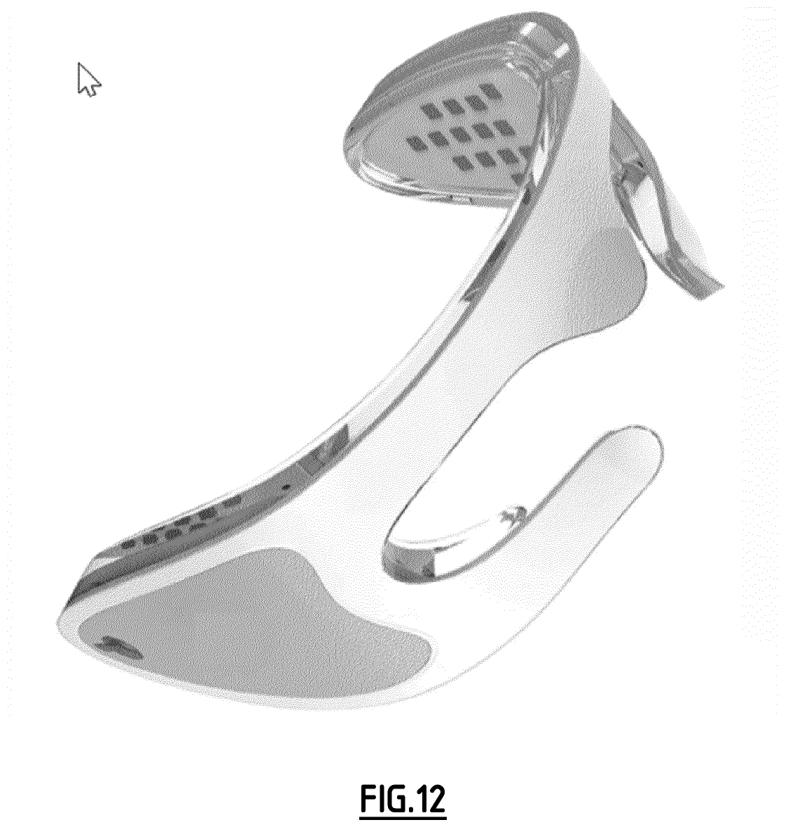

For applications in accordance with embodiments of the present disclosure, modules, such as modules as described in U.S. Patent Publication No. 2020/0253822, filed Mar. 18, 2020, and entitled “Module and Device for Emitting Electromagnetic Waves”, may be placed on an inside surface (i.e., a surface facing the human skin when the device is in its proper orientation) of a face mask (e.g., a facemask as depicted in the schematic diagrams of FIGS. 6 and 7), on the inside surface of a device suitable for applying millimeter wave RF stimulation to a human neck and/or décolletage (e.g., a device as depicted in the schematic diagram of FIG. 8), on the inside surface of a device suitable for applying millimeter wave RF stimulation to human lips (e.g., a device as depicted in the schematic diagram of FIG. 9), an inside surface of a device suitable for applying millimeter wave RF stimulation to human hands (e.g., a device as depicted in the schematic diagram of FIGS. 10 and 11), or an inside surface of a device suitable for applying millimeter wave RF stimulation to the eye region of a human face (e.g., a device as depicted in the schematic diagram of FIG. 12) in order to irradiate the desired areas. In accordance with embodiments of the disclosure, there may be one millimeter wave module or several millimeter wave modules per area. In accordance with embodiments of the disclosure, the millimeter wave modules may be driven in parallel. In embodiments, the millimeter wave modules may be driven sequentially in order to optimize comfort. By way of example only, FIG. 4 illustrates a module control unit in communication with a facemask having a plurality of modules disposed on an inside surface thereof that are designed to be stimulated sequentially. In embodiments, custom millimeter wave modules may be designed to perfectly fit the different treatment areas, with different sizes and shapes for each treatment location.

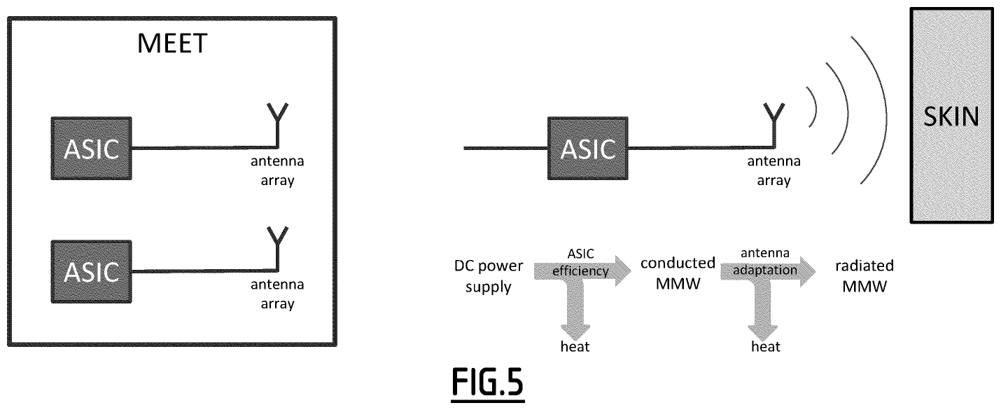

As both the transmitted millimeter wave RF energy and the conducted heat generated through the power consumption of the ASIC contribute to the desired skin response, it is possible to change the driving parameters of the ASIC in order to change its efficiency, hence changing the conducted heat/radiated RF energy ratio, for reaching the optimal treatment efficiency, which may rely on personalized conducted heat/radiated heat ratios. FIG. 5 is a schematic diagram illustrating an exemplary device wherein each millimeter wave module includes multiple ASICs. (It will be understood, however, that each millimeter wave module may include a single ASIC, if desired.) The driving parameters of each ASIC may be set by an external microcontroller that is capable of adjusting the ratio conducted heat/radiated RF energy, and total energy produced.

On the device itself or through a remote interface such as a smartphone app, the user can choose and adjust treatment parameters, such as: (1) area to be treated; (2) treatment duration; and (3) personal comfort settings. Treatment parameters also could be adjusted by a skin care professional remotely, following a diagnostic. The application to the face in the form of a face mask is a practical example, but could also be extended to gloves for treating the hands, or even clothing for treating other body parts.

Safety of the device would be ensured by using one or more temperature sensors within the mask or wearable. The temperature sensor could be used to ensure a maximum safety temperature is not exceeded, but also to optimize the desired ratio of conducted/radiated thermal energy.

Moreover, several thermal management strategies, such as the ones disclosed in patent application No. WO 2020/178111A1, using thermal spreaders of different conductivities and phase change materials could be used to increase efficiency of the therapy and comfort for the user.

Additionally, the proposed treatment could be used in conjunction with a skin care cream preparing the skin for receiving the treatment and optimizing its effect.

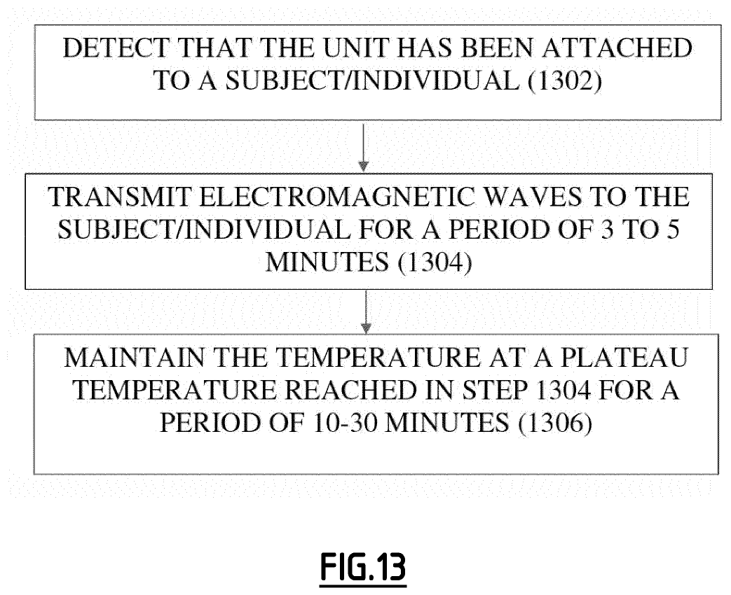

FIG. 13 illustrates a flow chart of an example process 1300 for treating decreases in skin volume and elasticity, according to certain aspects of the disclosure. In said process, the transmission of electromagnetic waves is performed for 3 to 5 minutes, but this is illustrative only. For explanatory purposes, some blocks of the example process of FIG. 13 are described herein as occurring in series, or linearly. However, multiple blocks of the example process of FIG. 13 may occur in parallel. In addition, the blocks of the example process of FIG. 13 need not be performed in the order shown and/or one or more of the blocks of the example process of FIG. 13 need not be performed.

At block 1302, it is detected that a device suitable for applying millimeter wave stimulation to a desired area of human skin is placed on a subject. For example, the unit may include any of the devices illustrated in FIGS. 6-12.

At block 1304, the device is activated such that it transmits millimeter wave stimulation to the subject during a first session such that there is a rapid rise in temperature for a few minutes, e.g., 3-5 minutes. For example, the subject may wear a device as depicted in FIG. 6 on their face, and the device may transmit millimeter waves through millimeter wave emitters. In an implementation, the device may have a power flux density of at least 0.5 milliwatts per square centimeter of skin and a frequency value between 3 and 120 gigahertz.

At block 1306, a temperature plateau may be held for a longer period (e.g., few tens of minutes) before allowing the skin to cool down again.

Antennas applied to the surface of the skin allow the skin temperature to be specifically increased in depth up to 1 mm and so stimulate the dermis area where the fibroblasts are located and the epidermis basal layer. This stimulation allows an increase in collagen synthesis and cell proliferation leading to a thickening of the skin. The millimeter wave frequencies disclosed herein induce a significant increase in the temperature at the fibroblast location, without damaging tissue or inducing painful sensations as may be observed with a direct heat source application or by using other electromagnetic waves frequencies or power densities.

Moreover, a process in accordance with FIG. 13 may allow for the activation of the response pathways to thermal stress initiating an adaptive response and stimulating antioxidant mechanisms to protect cells.

The invention also relates to a computer-implemented method for treating skin volume and laxity is disclosed that includes detecting that a device suitable for applying millimeter wave stimulation to a desired area of human skill is placed on a subject; activating the device such that millimeter wave stimulation is transmitted (e.g., through a transmitter of the device) to the subject during a first session lasting between 3 and 5 minutes, and maintaining the temperature for a period of between 10 and 30 minutes.

The invention also relates to a non-transitory computer-readable medium is disclosed that stores instructions that, when executed by a processor, cause the processor to perform a method for treating skin laxity and volume that includes detecting that a device suitable for applying millimeter wave stimulation to a desired area of human skill is placed on a subject; activating the device such that millimeter wave stimulation is transmitted (e.g., through a transmitter of the device) to the subject during a first session lasting between 3 and 5 minutes, and maintaining the temperature for a period of between 10 and 30 minutes.

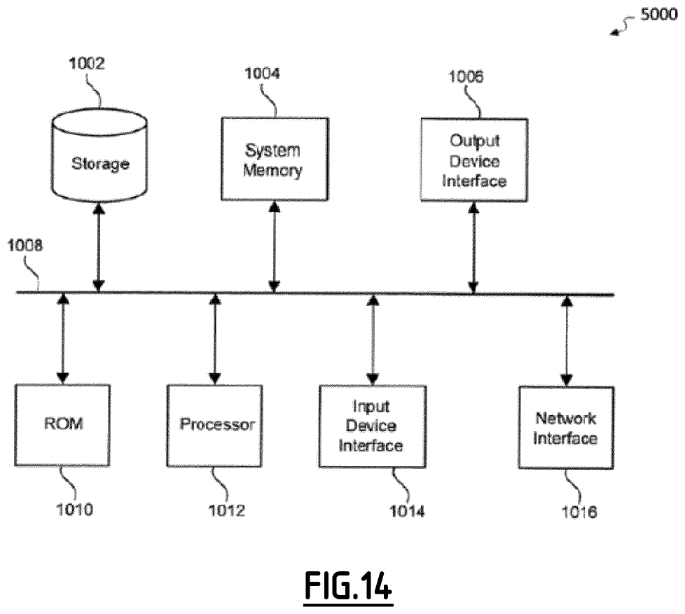

FIG. 14 conceptually illustrates electronic system 5000 with which one or more aspects of the subject technology may be implemented. Electronic system 5000, for example, may be, or may be a part of, a portable electronic device such as a laptop computer, a tablet computer, a phone, a wearable device, a personal digital assistant (PDA), a vehicle, or generally any electronic device that transmits signals over a network. Such an electronic system includes various types of computer-readable media and interfaces for various other types of computer-readable media. Electronic system 5000 includes bus 1008, processing unit(s) 1012, system memory 1004, read-only memory (ROM) 1010, permanent storage device 1002, input device interface 1014, output device interface 1006, and network interface 1016, or subsets and variations thereof.

Bus 1008 collectively represents all system, peripheral, and chipset buses that communicatively connect the numerous internal devices of electronic system 5000. In one or more embodiments, bus 1008 communicatively connects processing unit(s) 1012 with ROM 1010, system memory 1004, and permanent storage device 1002. From these various memory units, processing unit(s) 1012 retrieves instructions to execute and data to process in order to execute the processes of the subject disclosure. The processing unit(s) can be a single processor or a multi-core processor in different embodiments.

ROM 1010 stores static data and instructions that are needed by processing unit(s) 1012 and other modules of the electronic system. Permanent storage device 1002, on the other hand, is a read-and-write memory device. This device is a non-volatile memory unit that stores instructions and data even when electronic system 5000 is off. One or more embodiments of the subject disclosure uses a mass-storage device (such as a magnetic or optical disk and its corresponding disk drive) as permanent storage device 1002.

Other embodiments use a removable storage device (such as a floppy disk, flash drive, and its corresponding disk drive) as permanent storage device 1002. Like permanent storage device 1002, system memory 1004 is a read-and-write memory device. However, unlike storage device 1002, system memory 1004 is a volatile read-and-write memory, such as random access memory. System memory 1004 stores any of the instructions and data that processing unit(s) 1012 needs at runtime. In one or more embodiments, the processes of the subject disclosure are stored in system memory 1004, permanent storage device 1002, and/or ROM 1010. From these various memory units, processing unit(s) 1012 retrieves instructions to execute and data to process in order to execute the processes of one or more embodiments.

Bus 1008 also connects to input and output device interfaces 1014 and 1006. Input device interface 1014 enables a user to communicate information and select commands to the electronic system. Input devices used with input device interface 1014 include, for example, alphanumeric keyboards, pointing devices (also called “cursor control devices”), cameras or other imaging sensors, or generally any device that can receive input. Output device interface 1006 enables, for example, the display of images generated by electronic system 5000. Output devices used with output device interface 1006 include, for example, printers and display devices, such as a liquid crystal display (LCD), a light emitting diode (LED) display, an organic light emitting diode (OLED) display, a flexible display, a flat panel display, a solid state display, a projector, or any other device for outputting information. One or more embodiments may include devices that function as both input and output devices, such as a touch screen. In these embodiments, feedback provided to the user can be any form of sensory feedback, such as visual feedback, auditory feedback, or tactile feedback; and input from the user can be received in any form, including acoustic, speech, or tactile input.

Finally, as shown in FIG. 14, bus 1008 also couples electronic system 5000 to a network (not shown) through network interface 1016. In this manner, the computer can be a part of a network of computers (such as a local area network (“LAN”), a wide area network (“WAN”), or an Intranet, or a network of networks, such as the Internet. Any or all components of electronic system 5000 can be used in conjunction with the subject disclosure.

Many of the above-described features and applications may be implemented as software processes that are specified as a set of instructions recorded on a computer-readable storage medium (alternatively referred to as computer-readable media, machine-readable media, or machine-readable storage media). When these instructions are executed by one or more processing unit(s) (e.g., one or more processors, cores of processors, or other processing units), they cause the processing unit(s) to perform the actions indicated in the instructions. Examples of computer-readable media include, but are not limited to, RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, ultra-density optical discs, any other optical or magnetic media, and floppy disks. In one or more embodiments, the computer-readable media does not include carrier waves and electronic signals passing wirelessly or over wired connections, or any other ephemeral signals. For example, the computer-readable media may be entirely restricted to tangible, physical objects that store information in a form that is readable by a computer. In one or more embodiments, the computer-readable media is non-transitory computer-readable media, computer-readable storage media, or non-transitory computer-readable storage media.

In one or more embodiments, a computer program product (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

While the above discussion primarily refers to microprocessor or multi-core processors that execute software, one or more embodiments are performed by one or more integrated circuits, such as application specific integrated circuits (ASICs) or field programmable gate arrays (FPGAs). In one or more embodiments, such integrated circuits execute instructions that are stored on the circuit itself.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way), all without departing from the scope of the subject technology.

It is understood that any specific order or hierarchy of blocks in the processes disclosed is an illustration of example approaches. Based upon implementation preferences, it is understood that the specific order or hierarchy of blocks in the processes may be rearranged, or that not all illustrated blocks be performed. Any of the blocks may be performed simultaneously. In one or more embodiments, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

The subject technology is illustrated, for example, according to various aspects described above. The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

1. Cosmetic process for preventing and/or reducing the signs of skin aging and/or the resultant appearance of aged skin, and/or for preventing and/or reducing the signs of cutaneous stress, which comprises the use of a device capable of transmitting electromagnetic waves having a power flux density of at least 0.5 mW/cm² to skin and a frequency value of between 30 and 90 gigahertz.

2. The cosmetic process of claim 1, for preventing and/or reducing skin wrinkles and/or fine lines, withered skin, lack of elasticity and/or skin tone, thinning of the dermis and/or breakdown of collagen fibers resulting in the appearance of flabby, wrinkled skin, a thinning of an epidermis and/or a loss of firmness, elasticity, density and/or tone of an epidermis.

3. The cosmetic process according to claim 1, wherein the waves have a power flux density of between 0.5 and 15 mW/cm.

4. The cosmetic process according to claim 1, wherein the waves have a frequency value between 60 and 80 gigahertz.

5. The cosmetic process according to claim 1, wherein the device is capable of simultaneously exposing at least 1.25 cm² of the skin to the waves.

6. The cosmetic process according to claim 1, wherein the skin is human skin.

7. The cosmetic process according to claim 1, which comprises the use of a device comprising a detection unit of human or animal skin, and a transmitter, said process comprising the following steps:
a) the detection unit detects human or animal skin, and
b) when the unit detects that the skin is located at three millimeters or less from the transmitter, the transmitter transmits the electromagnetic waves, wherein said waves have a power flux density of at least 0.5 mW/cm² to skin and a frequency value of between 30 and 90 gigahertz.

8. The cosmetic process according to claim 7, wherein the device is suitable for applying electromagnetic waves to human skin in selected areas.

9. The cosmetic process according to claim 7, wherein the device is suitable for applying electromagnetic waves to human skin in at least one area chosen from the face, the hands, the décolletage, the eye region, the lips and the neck.

10. The cosmetic process according to claim 7, which comprises, before step a), a step of determining the initial state of skin.

11. The cosmetic process according to claim 7, which comprises, after step b), a step c) of checking the final state of skin.

12. The cosmetic process according to claim 7, which comprises, before or after step b), the topical administration of a cosmetic product onto skin.

13. The cosmetic process according to claim 11, which comprises, after step c), the topical administration of a cosmetic product onto skin.

14. The cosmetic process according to claim 13, wherein the cosmetic product comprises at least one active agent chosen from hydrating agents, anti-ageing agents, firming agents and their mixtures.

15. The cosmetic process according to claim 7, which comprises, in step b), adjusting the ratio of conducted heat and radiated heat transferred to the skin.

16. The process of claim 3, wherein the power flux density is between 3 and 12 mW/cm² or between 5 and 10 mW/cm².
